(19) United States
(12) Patent Application Publication
(10) Pub. No.: US 2022/0387370 A1
(43) Pub. Date: Dec. 8, 2022

(54) METHODS OF REDUCING NEUROLOGICAL DAMAGE IN WILSON DISEASE PATIENTS

(71) Applicant: Alexion Pharmaceuticals, Inc., Boston, MA (US)

(72) Inventors: Thomas Plitz, Geneva (CH); Sabine Borchard, Neuherberg (DE); Hans Zischka, Neuherberg (DE)

(73) Assignee: Alexion Pharmaceuticals, Inc., Boston, MA (US)

(21) Appl. No.: 17/775,755

(22) PCT Filed: Nov. 20, 2020
(86) PCT No.: PCT/US2020/061680

§ 371 Date: May 10, 2022

Related U.S. Application Data
(60) Provisional application No. 62/938,585, filed on Nov. 21, 2019.
(60) Provisional application No. 63/086,768, filed on Oct. 2, 2020.

(51) Int. Cl. A61K 31/28; A61P 25/28

(52) U.S. Cl. CPC A61K 31/28; A61P 25/28

(57) ABSTRACT

This disclosure generally relates to methods of treating copper-induced neurological damage observed in copper metabolism-associated diseases or disorders. This disclosure relates to reducing the copper-induced neurological damage in Wilson disease (WD).

### CROSS-REFERENCE TO RELATED APPLICATIONS

This application claims the benefit of priority of U.S. Provisional Patent Application No. 62/938,585, filed Nov. 21, 2019, and U.S. Provisional Patent Application No. 63/086,768, filed Oct. 2, 2020, both of which are incorporated by reference in their entirety.

### BACKGROUND OF DISCLOSURE.

### Field of Invention

This disclosure generally relates to methods of treating copper-induced neurological damage observed in copper metabolism-associated diseases or disorders. This disclosure specifically relates to reducing the copper-induced neurological damage in Wilson disease (WD).

### Technical Background

In 1912, Samuel Alexander Kinnier Wilson reported a fatal neurological disease characterized by a progressive degeneration of the lenticular nucleus that additionally was associated with liver cirrhosis. Today, we know that WD is related to an impairment of the mostly liver-residing copper-transporting ATPase ATP7B. A current consensus holds that ATP78 defects can cause massive liver copper accumulation that might spillover into the circulation. In WD patients, excess copper is not tightly incorporated into the copper-bearing plasma protein ceruloplasmin, but potentially available for accumulation in peripheral organs, such as the brain. In WD patients, after years or even decades of accumulation, brain copper concentrations may reach up to 450 μg/g dry weight (vs. 7-60 μg/g dry weight in controls), considered to be a prime toxic condition that can cause brain lesions and neurologic symptoms (e.g. dysarthria and parkinsonism). Nevertheless, many aspects of the pathophysiology of neurologic WD are still circumstantial or unknown. Among them is the issue of how non-ceruloplasmin-bound copper (NCC) enters and accumulates in the brain.

In WD patients, blood copper is mainly bound to albumin and amino acids. Being an abundant plasma protein (35-50 g/L; 500-750 μM), albumin has a huge copper binding capacity and may bind up to five copper ions at pH 7.4. Thereat, a first copper ion is rather tightly bound to the N-terminus (K_{d}=0.9×10⁻¹² M-6.7×10⁻¹⁷ M), a second one to a multi-metal binding site (MBS) with intermediate affinity (K_{d}=1.91×10⁻⁷ M) and the remaining three copper ions are relatively loosely attached to presently uncharacterized sites (K_{d}=6.25×10⁻⁶ M). In this respect, the amino acid histidine may play a minor role due to its comparatively lower plasma concentration (≈100 μM) and intermediate copper affinity (K_{d}≈10⁻⁹). Thus, in plasma several binding partners for copper exist with either high capacity and/or high affinity. In WD patients, total blood copper concentrations of 0.5-16.6 μM have been determined, among them a relatively low concentration of 1-5 μM copper that may be exchangeable as determined by EDTA (K_{d}=1.26×10⁻¹⁶ M) chelation experiments.

This raises the issue of how excess brain copper accumulation may form. One mechanism may be a constant competition for and uptake of copper into the brain via the high-affinity transporter CTR1 (K_{d}≈10⁻¹⁴ M), possibly linked to an impaired copper re-export into the blood due to ATP7B defects. CTR1 and ATP7B are present at the blood-facing membrane of endothelial cells that form the blood-brain barrier (BBB) along with astrocytes and pericytes. Such competition at relatively low blood copper may explain the observed long clinical silence of even decades in neurologic WD patients. A further, not mutually exclusive possibility is that periods of copper-induced liver tissue demise may cause intense “blood copper pulses”, thereby causing brain copper accumulation and damage. In fact, it has been observed as clinically relevant that the severity of neurologic symptoms commonly fluctuates, sometimes during the same day and that symptoms may be exacerbated by stress, concurrent illnesses, or medications. Such brain damage may start at the BBB, which then may facilitate further unregulated copper entry into the brain. In agreement, Stuerenburg described disturbances of the BBB in neurologic Wilson disease patients as indicated by an increased ratio of albumin presence in cerebrospinal fluid vs. serum.

The current treatments for WD are the general chelator therapies D-penicillamine (DPA) and trientine, which chelate Cu and promote urinary Cu excretion, and zinc (Zn), which blocks dietary uptake of Cu through upregulation of intestinal metallothionein. Treatment of neurologic WD patients with DPA can lead to dramatic symptom worsening as reported in 19-52% of the patients, such as shortly upon treatment onset. Such neurological worsening is hardly reported in tetrathiomolybdate (TTM)-treated patients. As DPA has a lower copper-affinity (K_{d}=2.4×10⁻¹⁶ M) than TTM (K_{d}=2.3×10⁻²⁰ M), due to its tight binding, competition for copper in the blood may be diminished by the latter, possibly leading to lower BBB and/or brain damage.

Therefore, there remains a need for effective treatments of the copper-induced neurological damages associated with copper metabolism-associated diseases or disorders, both to reverse the already present copper-induced neurological damage and to provide a lasting protection from future copper-induced neurological damage.

### SUMMARY OF THE DISCLOSURE

The disclosure generally provides methods useful for treating a copper-induced neurological damage in a subject. For example, the copper-induced neurological damage may be associated with a specific disease or disorder, such as Wilson disease.

One aspect of the disclosure provides a method of treating a copper-induced neurological damage. Such method comprises administering to the subject a therapeutically effective amount of bis-choline tetrathiomolybdate as described herein. In certain embodiments of this aspect, the methods reduce copper-induced neurological damage.

In certain embodiments, the copper-induced neurological damage comprises one or more of: copper-induced cell toxicity, copper-induced blood-brain barrier damage, and copper-induced mitochondrial damage. Thus, in certain aspect, the disclosure also provides a method of reducing copper-induced cell toxicity in a subject in need thereof. Such method comprises administering to the subject a therapeutically effective amount of bis-choline tetrathiomolybdate as described herein.

Another aspect of the disclosure provides a method of reducing copper-induced blood-brain barrier damage in a subject in need thereof. Such method comprises administering to the subject a therapeutically effective amount of bis-choline tetrathiomolybdate as described herein.

Another aspect of the disclosure provides a method of reducing copper-induced mitochondrial damage in a subject in need thereof. Such method comprises administering to the subject a therapeutically effective amount of bis-choline tetrathiomolybdate as described herein.

Another aspect of the disclosure provides a method of reducing cellular copper content in a subject in need thereof. Such method comprises administering to the subject a therapeutically effective amount of bis-choline tetrathiomolybdate as described herein.

Another aspect of the disclosure provides a composition comprising a therapeutically effective amount of bis-choline tetrathiomolybdate for use in the methods as described herein.

Additional aspects of the disclosure will be evident from the disclosure herein.

### BRIEF DESCRIPTION OF THE DRAWINGS

The accompanying drawings are included to provide a further understanding of the methods of the disclosure, and are incorporated in and constitute a part of this specification. The drawings illustrate one or more embodiment(s) of the disclosure and, together with the description, serve to explain the principles and operation of the disclosure.

FIGS. 1A-1E show that ALXN1840 and DPA increase blood copper levels. FIGS. 1A and 1E show PET scans of wild-type rats either i.v. or i.p. injected with ⁶⁴Cu. Intravenous injections resulted in a high signal intensity in the proximity of the brain, whereas no ⁶⁴Cu signal was detectable in i.p. injected rats. FIG. 1B shows the treatment of Atp7b^{-/-} with ALXN1840 for 4 consecutive days resulted in increased serum copper levels after sacrificing, whereas serum copper levels were unchanged after treatment with DPA (N=3). FIG. 1C shows treatment of Atp7b^{-/-} rats with DPA resulted in a significant increase in urinary copper excretion during treatment (N=3). FIG. 1D shows fecal copper excretion was unchanged in ALXN1840 and DPA treated animals during treatment (N=3). One-way ANOVA with Dunnett's multiple comparison test was used for statistical analysis. *p<0.05, **p<0.01, ***p<0.001, ****p<0.0001.

FIGS. 2A-2C show that ALXN1840 forms a stable complex with albumin and copper. FIG. 2A shows size-exclusion chromatography of 750 μM of copper and 250 μM albumin demonstrating the formation of a copper-albumin complex as well as a second copper peak representing unbound copper. In the presence of ALXN1840, albumin, molybdenum and copper were present in the same fractions. In the presence of DPA instead of ALXN1840, the distribution of copper was unchanged. (N=2). FIG. 2B shows the analysis of the contents of the Sudlow site I (SsI) in the structure of albumin. FIG. 2B, left panel, presents overall structure of albumin with indicated Sudlow site I. FIG. 2B, upper right panel presents a close-up of SsI with calculated difference map before refinement. Difference map indicate presence of additional molecules in this region. FIG. 2B, lower right panel presents the same site of SsI after refinement. ALXN1840 and copper atoms are covered by calculated 2F_{obs}−F_{calc} map indicating presence of these molecules inside SsI. FIG. 2C shows that electron paramagnetic resonance measurements revealed partial reduction of copper in the tripartite complex consisting of albumin, copper and ALXN1840. However, complete reduction of copper was only achieved by excess sodium dithionite (Na₂S₂O₄).

FIGS. 3A-3D show that ALXN1840 prevented HepG2, EA.hy926, U87MG and SHSY5Y cells from copper toxicity. All investigated cell lines showed a dose-dependent decrease in CellTiterGlo®-assessed cell viability by treatment with 250 μM albumin and increasing amounts of copper ranging from 250 to 2500 μM. In the presence of 750 μM DPA, this cytotoxic effect was not significantly increased compared to Cu-albumin alone. In contrast, the presence, of 750 μM ALXN1840 protected mainly HepG2, EA.hy926 and U87MG cells from cell death (N=3-5, n=6-10). Two-way ANOVA with Dunnett's multiple comparison test was used for statistical analysis. *p<0.05, **p<0.01, ***p<0.001, ****p<0.0001. FIGS. 3E-3H show that a low-dose of ALXN1840 partially rescued copper toxicity. A low dose of ALXN1840 (250 μM) partially rescued Cu-albumin-induced cell toxicity in HepG2 and EA.hy926 cells assessed by CellTiterGlo® assay. In contrast, low dose of DPA showed no effect on cell viability compared to cells treated with copper and BSA alone (N=3-5, n=6-10). Two-way ANOVA with Dunnett's multiple comparison test was used for statistical analysis. *p<0.05, **p<0.01, ***p<0.001, ****p<0.0001.

FIGS. 4A-4B show that high-affinity chelators reduced cell viability by cellular de-coppering. FIG. 4A shows that all investigated cell lines showed a dose-dependent reduction in CellTiterGlo®-assessed cell viability in the presence of the high-affinity chelator ALXN1840, which is not observed in the presence of DPA (N=3, n=6). FIG. 4B shows that complex IV activity was already reduced at a non-toxic ALXN1840 concentration (200 μM) in all cell lines, whereas 200 μM DPA had no effect on complex lV activity (N=3-4, n=6-10).

FIG. 5 shows that ALXN1840 led to a decreased cellular copper content in EA.hy926 and U87MG cells. FIG. 5, left panel shows that incubation with 750 μM copper and 250 μM albumin led to a massive accumulation of the metal in all investigated cell lines. In the presence of ALXN1840, U87MG and EA.hy926 cells presented with a (significantly) lower copper content compared to Cu-albumin alone. However, this effect was not present for HepG2 and SHSY5Y cells. In contrast, the presence of DPA did not change the amount of copper uptake in all cell lines (N=4-12). One-way ANOVA with Dunnett's multiple comparison test was used for statistical analysis. FIG. 5, right panel shows the Trypan blue-assessed cell viability of HepG2, EA.hy926, U87MG and SHSY5Y significantly decreased in the presence of 750 μM copper and 250 μM albumin. 750 μM ALXN1840, but not DPA, protected all cell lines from copper-related cell toxicity (N=4-12). One-way ANOVA with Sidak's multiple comparison test was used for statistical analysis. *p<0.05, **p<0.01, ***p<0.001, ****p<0.0001.

FIGS. 6A-6B show cellular parameters of EA.hy926 and U87MG cells subjected to high-resolution respirometry measurements. FIG. 6A shows cell viability assessed by Trypan blue (N=5-13), cell size (N=5-13) and cellular protein (N=3, n=9) content of EA.hy926 and U87MG cells were unchanged in untreated and Cu-albumin-treated cells in the absence or presence of ALXN1840 or DPA, respectively. In contrast, cellular copper content was significantly lower in Cu-albumin-treated EA.hy926 and U87MG cells in the presence of ALXN1840 compared to cells treated with Cu-albumin alone (N=5-13). FIG. 6B shows that mitochondrial respiration was decreased in EA.hy926 and U87MG cells in the presence of Cu-albumin and was significantly increased in the presence of ALXN1840 in EA.hy926 cells (N=4-7; ETS, capacity of the electron transport system). Two-way ANOVA with Dunnett's multiple comparison test was used for statistical analysis, *p<0.05, **p<0.01, ***p<0.001, ****p<0.0001.

FIGS. 7A-7B show copper-induced mitochondrial alterations can be protected by ALXN1840, but not DPA. FIG. 7A shows that mitochondrial structure of EA.hy926 and U87MG cells was altered in the presence of Cu-albumin. In the presence of ALXN1840, these alterations were partially reversed, whereas DPA had no positive effect on mitochondrial structure in both cell lines. Scale bars equal 500 nm. FIG. 7B shows respiratory control ratios (RCR) defined as routine to leak respiration (R/L) or ETS to leak respiration (E/L). In EA.hy926, E/L ratio was significantly increased in ALXN1840-treated compared to Cu-albumin treated cells. This effect was less pronounced in U87MG cells (N=4-7). Two-way ANOVA with Dunnett's multiple comparison test was used for statistical analysis. *p<0.05, **p<0.01, ***p<0.001, ****p<0.0001.

FIGS. 8A-8C show that non-toxic copper concentrations led to reduced TEER values of PBCEC monolayers. FIG. 8A shows exemplary curves of transendothelial electrical resistance (TEER) and capacitance changes of porcine brain cerebral endothelial cell (PBCEC) monolayers in the presence of increasing Cu-albumin (copper:albumin ratio of 1:3). The TEER decreased in a dose-dependent manner, whereas an increase in capacitance was only detectable at the highest. Cu-albumin concentration. FIG. 8B shows that neutral red assay of PBCECs revealed no toxicity below 250 μM copper (and 83.3 μM albumin) after 48 h. However, 750 μM copper (and 250 μM albumin) led to a dramatic reduction of cell viability, which could be rescued by the presence of 750 μM ALXN1840 (N=3, n=12). FIG. 8C show that the capacitance values of PBCEC monolayers were unaffected by Cu-albumin treatment in the absence or presence of ALXN1840 or DPA (N=2, n=4).

FIGS. 9A-9B show that copper-induced blood brain barrier damage can be rescued by ALXN1840, but not DPA. FIG. 9A shows Cu-albumin (250 μM copper and 83.3 μM albumin) led to a reduction in the transepithelial electrical resistance (TEER) of primary porcine brain capillary endothelial cell (PBCEC) monolayers. This could be rescued by 250 μM ALXN1840, respectively, whereas the presence of DPA led to a reduction of the TEER comparable to Cu-albumin treatment alone (N=2, n=4). FIG. 9B shows that determination of copper in the basolateral compartment (resembling the brain parenchyma) showed a significantly lower amount of copper in this compartment in the presence of the high-affinity chelator ALXN1840 after 48 h (N=2, n=4). Two-way ANOVA with Dunnett's multiple comparison test was used for statistical analysis. *p<0.05, **p<0.01, ***p<0.001, ****p<0.0001.

FIG. 10 shows that Cu-albumin leads to a disruption of tight junctions in PBCEC monolayers. Immunocytochemistry staining against the tight junction protein claudin-5 showed a continuous staining of the cell margins in control cells. This pattern was disrupted in primary porcine brain capillary endothelial cells (PBCECs) treated with Cu-albumin (250 μM copper and 83.3 μM albumin). In the presence of 250 μM ALXN1840, these morphologic alterations were less pronounced, whereas the presence of DPA did not prevent copper-induced gap formation. Staining against the tight junction-associated protein Zonula occludens-1 (ZO-1) revealed less distinct differences between control and Cu-albumin-treated cells, the latter being characterized by an uneven cytosolic distribution of ZO-1. This was prevented by ALXN1840, but still present in DPA-treated PBCEC monolayers. Scale bars equal 10 μm. Electron micrographs of copper-treated PBCECs revealed an impact of the metal on tight junction integrity as shown by a loss of electron-dense protein complexes at the cell-cell borders which can be prevented by ALXN1840, but not DPA. Scale bars equal 250 nm.

### DETAILED DESCRIPTION

Before the disclosed processes and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

In view of the present disclosure, the methods and compositions described herein can be configured by the person of ordinary skill in the art to meet the desired need. In general, the disclosed methods provide improvements in treatment copper-induced neurological damage observed in copper metabolism-associated diseases or disorders.

Thus, one aspect of the disclosure provides a method for reducing copper-induced neurological damage in a subject in need thereof.

In certain embodiments, reducing the copper-induced neurological damage improves, reduces, or eliminates one or more neurological or psychiatric symptoms. For example, the neurological symptoms that may be improved, reduced, or eliminated include, but not limited to, tremor, dysarthria, dystonia, and gait abnormalities. The psychiatric symptoms that may be improved, reduced, or eliminated include, but are not limited to, depression, social anxiety disorder, panic disorder, post-traumatic stress disorder, impairment of frontal-executive ability and/or visuospatial processing, and memory loss.

The reducing of copper-induced neurological damage may occur within a time interval ranging from day 0 to week 24 post administration. For example, the improvement, reduction, or elimination one or more neurological or psychiatric symptoms may occur within a time interval ranging from day 0 to week 24 post administration. In certain embodiments, the highest level of reduction of the copper-induced neurological damage may be within a time interval ranging from day 0 to week 7 post administration.

The copper-induced neurological damage may be presented as one or more of: copper-induced cell toxicity, copper-induced blood-brain barrier damage, and copper-induced mitochondrial damage. Thus, in certain embodiments, reducing copper-induced neurological damage is by reducing copper-induced cell toxicity, copper-induced blood-brain barrier damage, and/or copper-induced mitochondrial damage.

Certain embodiments of the methods of the disclosure include reducing copper-induced cell toxicity. For example, the copper-induced cell toxicity may be reduced in at least one of liver cells, endothelial cells, neurons, and astrocytes. The copper-induced cell toxicity may be reduced by at least 10%, e.g., by at least 15%, or by at least 20%, or by at least 25%, or by at least 30%, or by at least 35%, or by at least 40%, or by at least 45%, or by at least 50%, or even by at least 100%, relative to the copper-induced cell toxicity without bis-choline tetrathiomolybdate administration.

In certain embodiments, reducing copper-induced cell toxicity improves cell viability, as compared to the cell viability without bis-choline tetrathiomolybdate administration. For example, the cell viability may be improved by at least 10%, e.g., by at least 15%, or by at least 20%, or by at least 25%, or by at least 30%, or by at least 35%, or by at least 40%, or by at least 45%, or by at least 50%, or even by at least 100%.

In certain embodiments of the methods of the disclosure include reducing copper-induced blood-brain barrier damage. The copper-induced blood-brain barrier damage may be reduced by at least 10%, e.g., by at least 15%, or by at least 20%, or by at least 25%, or by at least 30%, or by at least 35%, or by at least 40%, or by at least 45%, or by at least 50%, or even by at least 100%, relative to the copper-induced blood-brain barrier damage without bis-choline tetrathiomolybdate administration.

In certain embodiments, the copper-induced blood-brain barrier damage may be reduced by one or more of: (a) maintaining transepithelial electrical resistance (TEER), (b) maintaining capacitance, and (c) reducing copper-induced morphological alterations For example, in certain embodiments, TEER is maintained at the same level as in a healthy subject. Likewise, in certain embodiments, capacitance is maintained at the same level as in a healthy subject. In certain embodiments, for example, the copper-induced morphological alteration is: a loss or structural disorientation of mitochondrial cristae and/or membranous inclusions, and/or continuous and uninterrupted presence of Claudin-5 and/or Zonula Occludens-1 presence at the cell borders of brain capillary endothelial cells.

Certain embodiments of the methods of the disclosure include reducing copper-induced neurological damage is by reducing copper-induced mitochondrial damage. For example, in certain embodiments, reducing of the copper-induced mitochondrial damage comprises one or more of: (a) reducing the presence of copper-induced membrane inclusions, (b) increasing or maintaining cristae organization, (c) increasing or maintaining electron-dense matrices, and (d) increasing or maintaining mitochondrial respiration, all as compared to untreated subjects.

In certain embodiments of the methods of the disclosure as described herein, the copper-induced neurological damage is associated with Wilson disease. For example, in certain embodiments, the subject suffers from Wilson disease.

The copper-induced neurological damage of the disclosure may result from another copper metabolism-associated disease or disorder. Thus, in certain embodiments, the copper metabolism-associated disease or disorder is copper toxicity (e.g., from high exposure to copper sulfate fungicides, ingesting drinking water high in copper, overuse of copper supplements, etc.). In certain embodiments, the copper metabolism associated disease or disorder is copper deficiency, Menkes disease, or aceruloplasminemia. In certain embodiments, the copper metabolism associated disease or disorder is at least one selected from academic underachievement, acne, attention-deficit/hyperactivity disorder, amyotrophic lateral sclerosis (ALS), atherosclerosis, autism, Alzheimer's disease, Candida overgrowth, chronic fatigue, cirrhosis, depression, elevated adrenaline activity, elevated cuproproteins, elevated norepinephrine activity, emotional meltdowns, fibromyalgia, frequent anger, geriatric-related impaired copper excretion, high anxiety, hair loss, hepatic disease, hyperactivity, hypothyroidism, intolerance to estrogen, intolerance to birth control pills, Kayser-Fleischer rings, learning disabilities, low dopamine activity, multiple sclerosis, neurological problems, oxidative stress, Parkinson's disease, poor concentration, poor focus, poor immune function, ringing in ears, allergies, sensitivity to food dyes, sensitivity to shellfish, skin metal intolerance, skin sensitivity, sleep problems, and white spots on fingernails.

As provided above, bis-choline tetrathiomolybdate (also known as ALXN1840, BC-TTM, tiomolibdate choline, tiomolibdic acid, and formerly known as WTX101) is administered in the methods of the disclosure. It has the following structure:

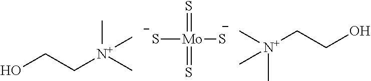

ALXN1840 is a first-in-class, Cu-protein binding agent in development for the treatment of WD and has been described in detail in International Publication No. WO 2019/110619 (incorporated by reference herein in its entirety). Prior work suggested that ALXN1840 improves control of Cu due to rapid and irreversible formation of Cu-tetrathiomolybdate-albumin tripartite complexes (TPCs). ALXN1840 monotherapy has been evaluated in 28 patients with WD, where it was shown that ALXN1840 reduced mean serum non-ceruloplasmin-bound Cu (NCC) by 72% at Week 24 compared with baseline. Treatment with ALXN1840 was generally well-tolerated, with most reported adverse events (AEs) being mild (Grade 1) to moderate (Grade 2). The most frequently reported drug-related AEs were changes in hematological parameters, fatigue, sulphur eructations, and other gastrointestinal symptoms. Reversible liver function test elevations were observed in 39% of patents; these elevations were mild to moderate, asymptomatic, were associated with no notable increases in bilirubin, and normalized with dose reduction or treatment interruption. No paradoxical neurological worsening was observed upon treatment initiation with ALXN1840.

In certain embodiments, reduction of copper-induced neurological damage is by formation of stable Cu-tetrathiomolybdate albumin tripartite complexes (TPCs) by ALXN1840. In certain embodiments, TPCs are available in the systemic circulation in the subject for transportation and/or elimination.

A therapeutically effective amount of ALXN1840 has been previously established. For example, in certain embodiments, ALXN1840 may be administered in the range of about 15 to 60 mg per day. In certain embodiments, ALXN1840 is administered in an amount of about 15 mg daily. In certain embodiments, ALXN1840 is administered in an amount of about 30 mg daily (e.g., about 15 mg taken twice daily or two 15 mg tablets taken once daily). In certain embodiments, ALXN1840 is administered in an amount of about 45 mg daily (e.g., about 15 mg taken trice daily or three 15 mg tablets taken once daily). In certain embodiments. ALXN1840 is administered in an amount of about 60 mg daily (e.g., about 15 mg taken four times daily or four 15 mg tablets taken once daily).

In certain other embodiments, ALXN1840 may be administered in the range of about 15 to 60 mg every other day. In certain embodiments, ALXN1840 is administered in an amount of about 60 mg every other day. In certain embodiments, ALXN1840 is administered in an amount of about 15 mg every other day. In certain embodiments, ALXN1840 is administered in an amount of about 30 mg every other day. In certain embodiments, ALXN1840 is administered in an amount of about 45 mg every other day. In certain embodiments, ALXN1840 is administered in an amount of about 60 mg every other day.

In certain embodiments of the present disclosure, increasing the therapeutically effective, amount of ALXN1840 during the treatment might provide additional benefits. Thus, in certain embodiments, the therapeutically effective amount of ALXN1840 is increased after 6 weeks (i.e., after 42 days) of treatment. For example, in certain embodiments, the initial therapeutically effective amount of ALXN1840 (i.e., days 1 to 42) is about 15 mg daily. The increased, subsequent therapeutically effective amount of ALXN1840 (i.e., after day 42, such as on day 43 and so on), in certain embodiments, is about 30 mg daily. In certain embodiments, the increased subsequent therapeutically effective amount of ALXN1840 is about 45 mg daily. In certain embodiments, the increased subsequent therapeutically effective amount of ALXN1840 is about 60 mg daily. For example, in certain other embodiments, the initial therapeutically effective amount of ALXN1840 is about 30 mg daily. The increased, subsequent therapeutically effective amount of ALXN1840, in certain embodiments, is about 45 mg daily. In certain embodiments, the increased subsequent therapeutically effective amount of ALXN1840 is about 60 mg daily.

In certain embodiments of the present disclosure decreasing the therapeutically effective amount of ALXN1840 during the treatment might provide additional benefits. Thus, in certain embodiments, the therapeutically effective amount of ALXN1840 is decreased after 6 weeks (i.e., after 42 days) of treatment. For example, in certain embodiments, the initial therapeutically effective amount of ALXN1840 (i.e., days 1 to 42) is about 60 mg daily. The decreased, subsequent therapeutically effective amount of ALXN1840 (i.e., after day 42, such as on day 43 and so on), in certain embodiments, is about 45 mg daily. In certain embodiments, the decreased subsequent therapeutically effective amount of ALXN1840 is about 30 mg daily. In certain embodiments, the decreased subsequent therapeutically effective amount of ALXN1840 is about 15 mg daily. For example, in certain other embodiments, the initial therapeutically effective amount of ALXN1840 is about 30 mg daily. The decreased, subsequent therapeutically effective amount of ALXN1840, in certain embodiments, is about 15 mg daily.

The methods of the disclosure are useful as a first line treatment. Thus, in certain embodiments of the methods of the disclosure, the subject previously received no treatment for Wilson disease (i.e., a treatment-nave subject).

The methods of the disclosure are also useful as a second line treatment and/or a first line maintenance treatment of WD. Thus, in certain embodiments of the methods of the disclosure, the subject has previously received a standard of care (SoC) treatment for WD. For example, in certain embodiments, the subject has previously received trientine (also known as triethylenetetramine; N′-[2-(2-aminoethylamino)ethyl]ethane-1,2-diamine). Trientine may be sold under the names CUPRIOR® (GMP-Orphan United Kingdom Ltd). SYPRINE® (Aton Pharma, Inc.), or Cufence (Univar, Inc.). In certain embodiments, the subject has previously received D-penicillamine (also known as penicillamine; (2S)-2-amino-3-methyl-3-sulfanylbutanoic acid). D-penicillamine may be sold under the names CUPRIMINE® (Valeant Pharmaceuticals) or DEPEN® (Meda Pharmaceuticals). In certain embodiments, the subject has previously received zinc. In certain embodiments, the subject has previously received trientine, D-penicillamine, and/or zinc. In certain other embodiments, the subject has previously received trientine and/or D-penicillamine.

In certain embodiments of the methods of the disclosure, the subject has received standard of care treatment for WD for no more than 24 weeks. In certain embodiments, the standard of care treatment was no more than 12 weeks, or no more than 6 weeks, or no more than 4 weeks. The standard of care treatment need not be continuous. For example, the subject may receive the treatment on-and-off totaling no more than 24 weeks (e.g., no more than 12 weeks, or no more than 6 weeks, or no more than 4 weeks) of treatment. In certain embodiments, however, the standard of care treatment is continuous.

In certain embodiments of the methods of the disclosure, the subject has received standard of care treatment for WD for no more than 4 weeks.

In certain embodiments of the methods of the disclosure, the subject has received standard of care treatment for WD for at least 4 weeks. In certain embodiments, the standard of care treatment was at least 6 weeks, or at least 12 weeks, or at least 24 weeks, or at least 36 weeks, or at least 48 weeks, or at least 52 weeks long. The standard of care treatment need not be continuous. For example, the subject may receive the treatment on-and-off totaling at least 4 weeks (e.g., at least 6, or at least 12, or at least 24, or at least 36, or at least 48, or at least 50 or at least 52 weeks or at least 103 weeks) of treatment. In certain embodiments, however, the standard of care treatment is continuous.

In certain embodiments of the methods of the disclosure, the subject previously received no treatment or the subject previously received a standard of care treatment for no more than 4 weeks for the copper metabolism-associated disease or disorder, such as for Wilson disease.

In the methods of the disclosure a described herein, the subject completed the standard of care treatment at least 2 weeks prior to administering bis-choline tetrathiomolybdate. In certain embodiments, the subject completed the standard of care treatment at least 3 weeks, at least 4 weeks, or at least 6 weeks prior to administering bis-choline tetrathiomolybdate.

As used herein, the terms “individual”, “patient,” or “subject” are used interchangeably, and refer to any animal, including mammals, and, in at least one embodiment, humans. In certain embodiments, the subject is a healthy subject. In certain embodiments, the subject suffers from WD. In certain embodiments of the methods of the disclosure as described herein, the subject has cirrhosis. In certain other embodiments, the subject does not have cirrhosis.

In certain embodiments, when values are expressed as approximations, by use of the antecedent “about,” it will be understood that the particular value forms one possible embodiment and variation of the given value is possible (e.g., about 80 may include 80±10%). It will be further understood that the endpoints of each of the ranges are significant both in relation to the other endpoint, and independently of the other endpoint.

As used herein, “total copper” refers to the sum of ail copper species in blood (for example, in serum or plasma). Total copper includes both ceruloplasmin (Cp)-bound copper and all species of non-ceruloplasmin bound copper. In general, total copper may be directly measured with high sensitivity and specificity by mass-spectroscopy, such as inductively coupled plasma-mass spectrometry (ICP-MS).

The term “NCC” refers to the fraction of total copper that is not bound to ceruloplasmin (i.e., “non-ceruloplasmin-bound copper”) and which is estimated using direct measurements of total copper and Cp in the blood (such as, e.g., serum or plasma) and the following formula:

$$NCC [ µ M ] = \frac{Total plasma Cu [ µg / L ] - ( 3.15 * ceruloplasmin [ \frac{mg}{L} ] )}{63.5 [ µ g / µmol ]}$$

The calculation is premised on an assumption that six copper atoms are always bound to a single Cp molecule, and that NCC and ceruloplasmin concentrations are directly correlated. In reality, Cp may show considerable heterogeneity in the number of copper atoms associated per Cp molecule. This formula assumes that six copper atoms bind per one Cp molecule, but the copper/Cp ratio varies with disease state. In fact, 6-8 copper atoms can actually bind to Cp, and in WD usually fewer than six copper atoms are associated per Cp molecule.

Non-ceruloplasmin-bound copper includes the fraction of total copper that is bound, to albumin, transcuprein, and other less abundant plasma proteins or in tetrathiomolybdate-Cu-albumin tripartite complexes (TPCs). The concentration of TPCs cannot be directly measured, but in certain embodiments, the concentration of TPCs may be estimated using molybdenum concentration as a surrogate.

### EXAMPLES

The methods of the disclosure is illustrated further by the following examples, which are not to be construed as limiting the disclosure in scope or spirit to the specific procedures and compounds described in them.

### Materials and Methods

MicroPET/MRI

Wild-type rats underwent anatomical magnetic resonance imaging (MRI) 1T and dynamic positron emission tomography (PET) (Mediso Medical Imaging Systems, Budapest, Hungary). Anesthesia with isoflurane was initiated with the rat placed in an acrylic glass chamber and maintained with respiration in a mask during the scan. A bolus of ⁶⁴Cu (≈10 MBq/animal) was injected via a tail vein catheter or intraperitoneal. PET scanning was performed the first 120 min after injection, followed by a 25 min T1-weighted MRI scan.

PET images were reconstructed with a three-dimensional ordered subset expectation algorithm (Tera-Tomo 3D; Mediso Medical Imaging Systems, Budapest, Hungary) with four iterations and six subsets and a voxel size of 0.6×0.6×0.6 mm³. Data was corrected for dead-time, decay, and randoms using delayed coincidence window without corrections for attenuation and scatter. The 120 min dynamic PET scans were reconstructed as 8 frames of 15 min.

Control Atp7b^{+/-} and WD Atp7b^{-/-} rats were fed ad libitum with normal chow (1314; 13.89 mg Cu/kg; Altromin Spezialfutter GmbH, Seelenkamp, Germany) and tap water. All rats were healthy at treatment start and presented no signs of acute liver damage (serum AST <200 U/L and serum bilirubin <0.5 mg/dl). Atp7b^{-/-} rats (age: 79-96 days) were treated intraperitoneally for 4 consecutive days with 2.5 mg/kg body weight (bw) bis-choline TTM (ALXN1840) once daily or 100 mg/kg bw D-penicillamine (DPA) once daily. Untreated Atp7b^{+/-} and Atp7b^{-/-} rats served as controls. Urine and feces were collected at 24-hour intervals for which rats were housed individually in metabolic cages for 4 days. After a two-day resting period off treatment in normal cages and group housing, rats were sacrificed for serum collection. Copper levels in urine, serum and feces were analyzed by inductively coupled plasma optical emission spectrometry (ICP-OES, ARCOS, SPECTRO Analytical Instruments, Kleve, Germany) as previously described (Zischka, H., et al., J Clint Invest, 2011, 121(4): p. 1508-18).

Gel Filtration Chromatography

10 mg of fatty acid free bovine serum albumin (subsequently referred to as albumin, Carl Roth, Karlsruhe, Germany) were resuspended in 10 mM Tris-HCl buffer (pH 7.4) and mixed with 45 μL of 10 mM copper chloride. Where indicated, 45 μL of 10 mM ALXN1840 or DPA were added to the Cu-albumin complex before loading the samples onto a Superdex 75 10/300 GL column (GE Healthcare, Chicago, USA). 1 mL fractions were analyzed for protein content by the Bradford assay (Bradford, M. M., Anal Biochem, 1976. 72: p. 248-54), molybdenum and copper levels by ICP-OES (Zischka, H., et al., J Clin Invest, 2011. 121(4): p. 1508-18) and DPA content using 1,2-naphthoquinone-4-sulfonate (NQS) as previously described (Elbashir, A., PharmacoVigilance Review, Vol. 01:2. 2013) with minor modifications. Briefly, 50 μL of each fraction were mixed with 10 μL 0.2% NQS, 10 μL of 0.2 M sodium phosphate buffer (pH 12.0) and 30 μL ddH₂O in a clear 96-well plate. The samples were incubated for 20 min and absorbance was measured at 452 nm. Absolute levels of DPA were calculated using equally treated DPA standard solutions (25 μM to 250 μM).

X-Ray Crystallography of the Tripartite Complex Albumin-Copper-ALXN1840 (TPC)

100 mg albumin were suspended in buffer containing 50 mM potassium phosphate and 150 mM sodium chloride (pH 7.5). Copper chloride and ALXN1840 were added in double molar excess and the mixture was incubated for 30 min at 37° C. The Cu-albumin-ALXN1840 mixture was loaded onto a S200 gel filtration column equilibrated with PBS (pH 7.4). Fractions corresponding to the Cu-albumin-ALXN1840 (TPC) protein in the monomeric state were pooled and protein was concentrated to 100 mg/mL. Screening for crystallization conditions was performed using commercially available buffer sets in a sitting-drop vapor diffusion setup by mixing 0.2 μL of protein complex solution and 0.2 μL of buffer solution. Crystals were obtained at room temperature from a solution containing 0.1 M SPG buffer (pH 7.0) and 0.25% PEG 1500. Crystals were cryo-protected in 30% glycerol in the mother liquor and flash-cooled in liquid nitrogen. The diffraction data were collected at the ID23-2 beamline at ESRF (Grenoble, France). The data were indexed and integrated using XDS (Krug, M., et al., Journal of Applied Crystallography, 2012. 45(3): p. 568-572; Kabsch, W., Acta Crystallogr D Biol Crystallogr, 2010. 66(Pt 2): p. 125-32), scaled and merged using Scala (Evans, P. Acta Crystallogr D Biol Crystallogr, 2006. 62(Pt 1): p. 72-82). The initial phases were obtained by molecular replacement calculated using Phaser (McCoy, A. J., Methods Mol Biol, 2017. 1607: p. 421-453) and albumin structure as a search model (PDB 4F5S and (Bujacz, A., Acta Crystallogr D Biol Crystallogr, 2012. 68(Pt 10): p. 1278-89). The initial model was manually rebuilt due to the resulting electron density maps using Coot (Emsley, P., et al., Acta Crystallogr D Biol Crystallogr, 2010. 66(Pt 4): p. 486-601). Due to the low resolution of data, refined structure did not reach R_{free} values below 0.40. Nevertheless, a final model in terms of presence of ALXN1840 was able to be analyzed due to the high scattering factor of the molybdenum complex resulting in a strong detectable signal.

Electron Paramagnetic Resonance (EPR)

For EPR measurements, complexes of Cu-albumin (2 mM/1 mM), Cu-albumin-ALXN1840 (TPC, 2 mM/1 mM/1 mM) and albumin-ALXN1840 (1 mM/1 mM) were prepared in 10 mM Tris HCl (pH 7.4) buffer and reduced with an excess of sodium dithionite (Merck, Darmstadt, Germany) shortly prior to measurements where indicated. EPR spectra were recorded at 77 K using an ECS106 spectrometer (Brucker BioSpin, Karlsruhe, Germany) operating in X-band at about 9.5 GHz.

Cell Culture

SHSY5Y (human neuroblastoma), U87MG (human astrocytoma), EA.hy926 (human endothelium) and HepG2 (human hepatocellular carcinoma) cells were from ATCC (Wesel, Germany) and were cultured in Dulbecco's Modified Eagle Medium (DMEM) supplemented with 10% FCS (Biochrom, Berlin, Germany) and 1% antibiotic-antimycotic (Life Technologies, Carlsbad, USA). All cells were maintained at 37° C. in a humidified atmosphere with 5% CO₂.

Cell Toxicity Assays

2×10⁴ cells were seeded into each well of a 96-well plate and incubated overnight. On the next day, cells were treated for 24 hours with increasing copper concentrations (0 to 2500 μM) and 250 μM albumin (resulting in Cu-albumin molar ratios of 1:1, 2:1, 3:1, 4:1 and 10:1) in the absence or presence of 750 μM ALXN1840 or DPA in DMEM (2% FCS). Cell toxicity was either determined by CellTiter-Glo® assay (Promega, Madison, USA) or Trypan blue exclusion test.

Cellular Copper Content

2×10⁶ cells were incubated for 24 hours with 750 μM copper and 250 μM albumin (i.e., at a Cu-albumin molar ratio of 3:1) in the absence or presence of 750 μM ALXN1840 or DPA, respectively, in DMEM (2% FCS). Afterwards, cells were trypsinized and counted. Cell viability was determined by Trypan blue exclusion test. Copper and molybdenum content of cells was analyzed by ICP-OES (Ciros Vision, SPECTRO Analytical Instruments, Kleve, Germany) as previously described (Zischka, H., et al., J Clin Invest, 2011. 121(4): p. 1508-18).

Electron Microscopy

Electron microscopy of cells was done as previously described (Einer, C., et al., Cell Mol Gastroenterol Hepatol, 2018. 7(3): p. 571-596) on a 1200EX electron microscope (JEOL, Akishima, Japan) at 60 kv. Pictures were taken with a KeenView II digital camera (Olympus, Hamburg, Germany) and processed by the iTEM software package (analySIS FIVE, Olympus, Hamburg, Germany).

Mitochondrial Function

U87MG and EA.hy926 cells were pretreated for 24 hours with DMEM (2% FCS) alone or with DMEM (2% FCS) containing 750 μM copper chloride and 250 μM albumin in the absence or presence of 750 μM ALXN1840 or DPA. Oxygen consumption was assessed by high-resolution respirometry (HRR) using the Oxygraph-2k and DatLab 7.0 (Oroboros Instruments GmbH, Innsbruck, Austria) as described previously (Pesta, D. and E. Gnaiger, Methods Mol Biol, 2012. 810: p. 25-58). Per chamber, 1.5-2×10⁶ living cells were supplied in 2 mL of MiR05 buffer (0.5 mM EGTA, 3 mM MgCl₂, 60 mM lactobionic acid, 20 mM taurine, 10 mM KH₂PO₄, 20 mM HEPES, 110 mM sucrose, 1 g/L albumin, pH 7.1) and routine respiration was measured. Addition of 2.5 μM oligomycin (inhibitor of the F_{O}F₁-ATPase) enabled the measurement of leak respiration and stepwise addition of CCCP (1 μL steps from 1 mM stock solution) allowed the determination of the maximum oxygen flux and thereby the capacity of the electron transport system (ETS). The oxygen flux was baseline-corrected for non-mitochondrial oxygen consuming processes (ROX) by the addition of 2.5 μM of the complex III-inhibitor antimycin A.

For complex IV activity measurements, cells were pretreated for 24 hours with DMEM (2% FCS) alone or with DMEM (2% FCS) containing 750 μM copper chloride and 250 μM albumin in the absence or presence of 750 μM ALXN1840 or DPA. Complex IV activity was measured as previously described (Spinazzi, M., et al., Nat Protoc, 2012. 7(6): p. 1235-46). Briefly, about 2.5×10⁶ cells were detached, washed two times with PBS by centrifugation and the cell pellet was resuspended in 200 μL of 20 mM hypotonic potassium buffer. After 3 freeze-thaw cycles, complex IV activity was measured by adding 10 μL of the sample to 90 μL of 50 mM potassium phosphate buffer (pH 7.0) containing 50 μM reduced cytochrome c with or without 0.3 mM KCN. Absorbance was measured at 550 nm for 10 min in a plate reader (Synergy 2, BioTek Instruments, Inc., Bad Friedrichshall, Germany) and complex IV activities were calculated from the linear slopes of the initial rates corrected for unspecific activity (in the presence of KCN) and normalized to the protein content determined by the Bradford assay.

Endothelial Blood-Brain Barrier Model

For transepithelial resistance (TEER) experiments (Srinivasan, B., et al., J Lab Autom, 2015. 20(2): p. 107-26), cryopreserved primary porcine brain capillary endothelial cells (PBCECs) were thawed and seeded either on rat tail collagen-coated 96-well plates for cytotoxicity testing using the Neutral red assay (Repetto, G., A. del Peso, and J. L. Zurita, Nat Protoc, 2008. 3(7): p. 1125-31) or Transwell® inserts (area: 1.12 cm², pore size: 0.4 μm: Corning, N.Y., USA) for barrier integrity studies. Cells were cultured for 48 hours in Earle's Medium 199 supplemented with 10% FCS, 50 U penicillin/mL, 50 μg/mL streptomycin, 100 μg/mL gentamycin and 0.7 mM I-glutamine and maintained at 37° C. in a humidified atmosphere with 5% CO₂. Subsequently, the medium was changed to DMEM/Ham's F 12 (1:1) containing 50 U penicillin/mL, 50 μg/mL streptomycin, 100 μg/mL gentamycin and 4.1 mM L-glutamine and 550 nM hydrocortisone for additional 48 hours upon which the medium was changed to the treatment solution containing 250 μM copper (and 83.3 μM albumin, Cu-albumin molar ratio 3:1) in the absence or presence of 250 μM DPA or ALXN1840, respectively. TEER and capacitance values were continuously monitored over 48 h using a CellZscope device (nanoAnalytics, Münster, Germany). Only PBCEC monolayers with initial TEER values >600Ωcm² and capacitance values between 0.45 and 0.6 μF/cm² were used for permeability studies (Bornhorst, J., et al., J Biol Chem, 2012. 287(21): p. 17140-51). The barrier integrity was calculated by normalizing the TEER values to the respective start values.

At treatment start, after approximately 24 and 48 h exposure to the test substances, 20 μL of the apical medium as well as 40 μL of the basolateral medium were collected for subsequent copper determination. Total copper was determined by ICP-MS/MS as previously described (Kopp, J. F., et al., J Trace Elem Med Biol, 2019. 54: p. 221-225). For the immunocytochemical staining of tight junction proteins, confluent PBCECs cultivated on Transwell® membrane inserts were processed as previously described (Müller, S. M., et al., Archives of Toxicology 2018. 92(2): p. 823-832). Briefly, PBCECs were fixed with formaldehyde and permeabilized using Triton X-100. After blocking of unspecific binding sites by albumin, the cells were incubated with the either anti-claudin 5 or anti-ZO-1 antibody (Zytomed Systems GmbH, Berlin, Germany). Following a second blocking step, the cells were treated with an Alexa Fluor® 488 conjugated secondary antibody (Invitrogen, Molecular Probes Inc., Eugene, USA). Hoechst 33258 (Merck, Darmstadt, Germany) was used to stain cell nuclei. Subsequently, membranes were cut out of the inserts and mounted in Aqua Poly/Mount (Polysciences Inc., Washington, USA). After a solidification period of 24 hours, the samples were evaluated using a DM6 B fluorescence microscope by Leica Microsystems CMS GmbH (Wetzlar, Germany) in combination with the Leica Application Suite X.

Electron microscopy of PBCECS grown on Transwell® inserts was performed as previously described (Ye, D., K. A. Dawson, and I. Lynch, Analyst, 2015. 140(1): p. 83-97) with minor modifications. Briefly, after fixation with 2.5% glutaraldehyde, cell monolayers were post-fixed with 1% osmium tetroxide for 30 min and dehydrated by ethanol. Cell monolayers were gradually embedded in epoxy resin in ethanol (1:2, 1:1, 2:1 for 20 min each) and finally embedded in 100% epoxy resin for 48 h at 60° C. without pre-embedding prior to cutting and image acquisition.

Miscellaneous/Statistics

Cellular protein levels were determined by the BCA assay (Smith, P. K., et al., Anal Biochem, 1985. 150(1): p. 76-85). Cell size was determined using a LUNA-II™ Automated Cell Counter (Logos biosystems, Anyang, South Korea).

Throughout this disclosure “N” designates the number of biological replicate and “n” the number of technical replicates. Data are mean values with standard deviation (SD). Statistical significance was analyzed with the respective tests indicated in the figure legends using GraphPad Prism 7 (GraphPad Software Inc., La Jolla, USA).

### Example 1

### Copper Chelators Elevate Blood Copper Differentially

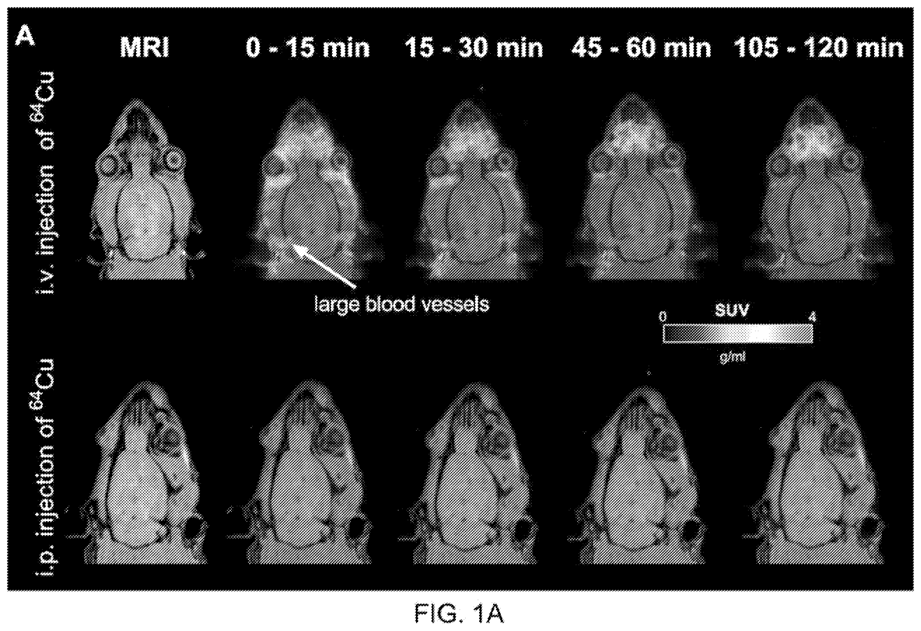

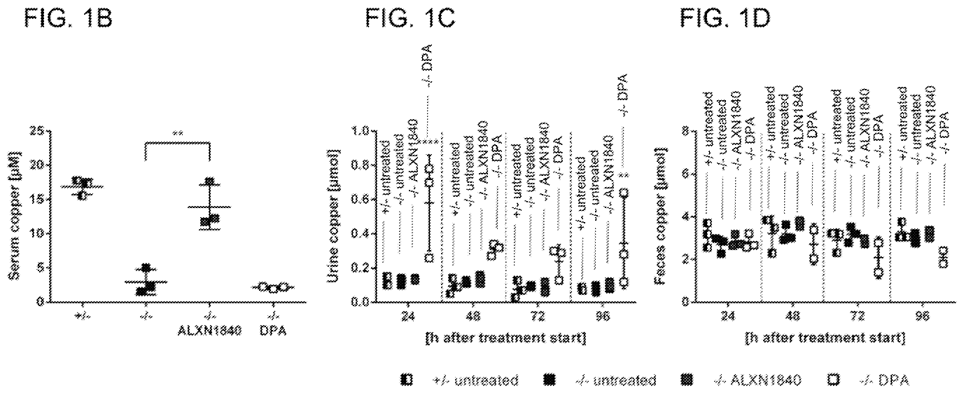

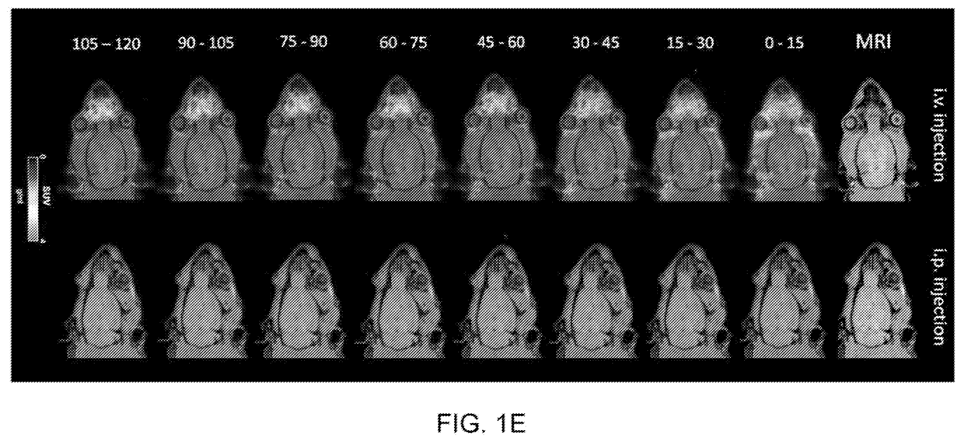

Chelation is a therapeutic approach in Wilson disease (WD). While mobilization of excess copper might be necessary for renal clearance of chelated copper, it could nevertheless lead to potentially undesirable systemic copper effects, e.g., “neurological worsening”. Indeed, upon intravenous ⁶⁴Cu-injection, within minutes the metal can be largely traced by PET in brain supporting vessels in wild-type rats (FIG. 1A). In contrast, intraperitoneal ⁶⁴Cu-injection, mimicking nutritional copper uptake, hardly, even after hours, increases copper there (FIGS. 1A and 1E).

It was investigated to what extent copper appears in serum in untreated Atp7b^{+/-} control and Atp7b^{-/-} rats (alternatively termed WD rats), but also in Atp7b^{-/-} rats treated with either DPA or ALXN1840 (FIG. 1B). As in WD patients, WD rats lack copper incorporation into ceruloplasmin, and therefore untreated Atp7b^{-/-} animals have a very low serum copper level. Upon ALXN1840 treatment, however, serum copper levels of WD rats were significantly increased (plausibly due to ALXN1840-Cu-albumin tripartite complex formation, see below), but not upon DPA treatment (FIG. 1B). As this latter absence may be due to fast renal copper clearance, the excretion of copper via urine (FIG. 1C) but also feces (FIG. 1D) was investigated. While untreated Atp7b^{+/-} and Atp7b^{-/-} rats had low copper levels in either urine or feces collected over 24 h, DPA treatment of WD rats led to a significantly increased copper excretion into urine, in agreement with typical diagnostic findings in WD patients. In contrast, no profoundly elevated net copper excretion was noted upon ALXN1840 treatment under the chosen conditions (i.e., an observation period of 96 hours). Thus, these chelators may elevate blood copper levels to a different extent. While in the case of DPA a rapid renal clearance (blood peak between 1 to 3 hours after application) may have avoided its detection, a significantly elevated copper serum level was observed upon ALXN1840 treatment in WD rats.

### Example 2

### ALXN1840 Forms a Stable Complex with Copper and Albumin

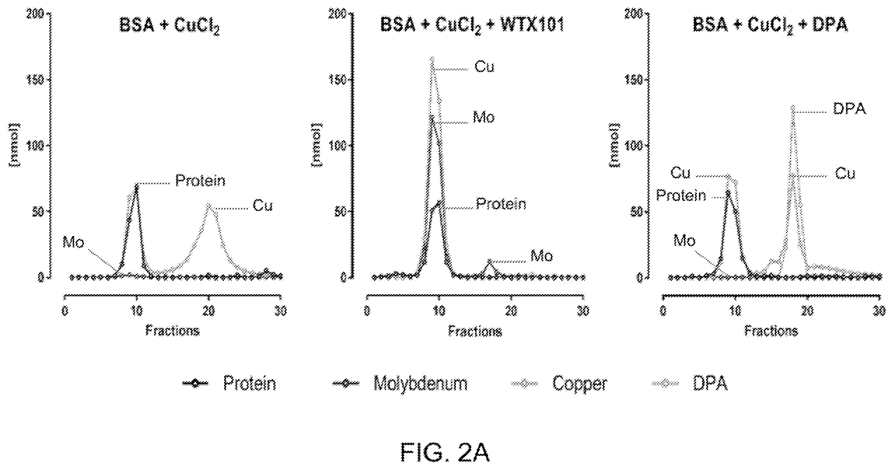

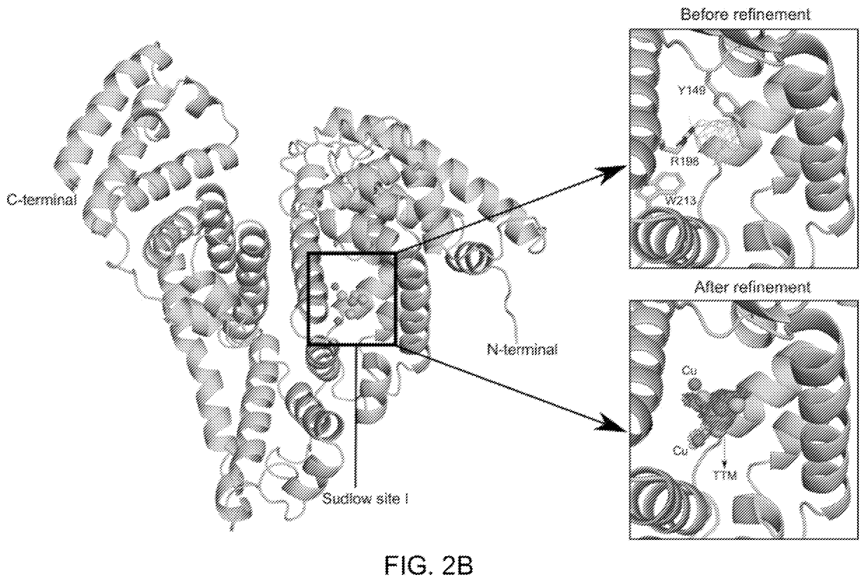

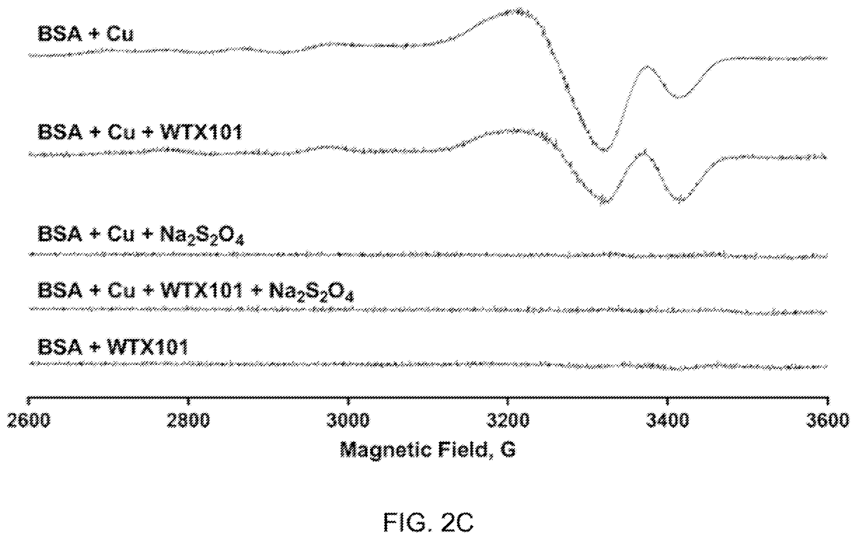

In WD patients, copper may be loosely-bound to albumin. In fact, upon mixing 750 μM copper with 250 μM albumin (i.e., a molar ratio 3:1), a subsequent gel filtration removed about half to two thirds of the copper from albumin (FIG. 2A top panel). When incubated with DPA (at a molar ratio Cu-albumin-DPA of 3:1:3), parts of the copper stayed with albumin, plausibly due to DPA's lower copper affinity in comparison to the high-affinity albumin binding site (K_{d}(DPA)=2.4×10⁻¹⁶ M vs. K_{d}(N-terminus of albumin)=6.7×10⁻¹⁷ M), whereas the residual copper co-migrated with DPA (FIG. 2A lower panel). It thus appears that the capacity of DPA to de-copper albumin might be limited to the loosely bound copper at the applied molar ratios.

Intriguingly, when co-incubated with the high-affinity chelator ALXN1840 (K_{d}=2.3×10⁻²⁰ M), albumin was not fully de-coppered but rather one prominent gel filtration peak appeared (FIG. 2A middle panel), comprising the protein and large parts of copper as well as ALXN1840 (detected as molybdenum). This feature has been described for TTM (TTM is the active molecule in ALXN1840) and is due to the formation of a Cu-albumin-ALXN1840/TTM complex, previously termed the “tripartite complex” (TPC).

The single TPC gel filtration peak indicated copper's tight binding to the TPC protein. Consequently, X-ray crystallography of the TPC was employed (FIG. 2B) and, upon data refinement, ALXN1840 was tracked with two bound copper ions in the so-called Sudlow Site 1, a profound cleft in albumin formed by His241, Tyr149, Arg256, Lys237, Ala290 (FIG. 2B). Thus, upon co-presence of albumin, copper and ALXN1840, the chelator-bound copper became deeply embedded into the protein. This finding may explain the lack of urinal copper excretion in ALXN1840-treated WD rats (FIG. 1C), as renal albumin clearance is very limited. In addition, electron paramagnetic resonance (EPR) studies demonstrated a change in copper redox status in the TPC versus Cu-albumin (FIG. 2C). While the latter revealed the typical cupric Cu(II) signal, in the presence of ALXN1840 the signal intensity of the EPR-active cupric copper dropped by around 50%, suggesting the reduction of one cupric copper ion to EPR-silent cuprous Cu(I) (FIG. 2C second trace). Indeed, upon addition of sodium dithionite that fully reduces Cu(II) to Cu(I), no EPR signal was detected in both Cu-albumin and TPC (FIG. 2C lower traces).

In summary, using the above molar ratios, only the tightly bound copper stayed with albumin, whereas more loosely-bound copper was either set free upon gel filtration, bound to DPA, or relocated to the Sudlow site of albumin by ALXN1840 forming the TPC.

### Example 3

### High-Affinity Chelation Prevents Cu-Albumin-Induced Cell Toxicity

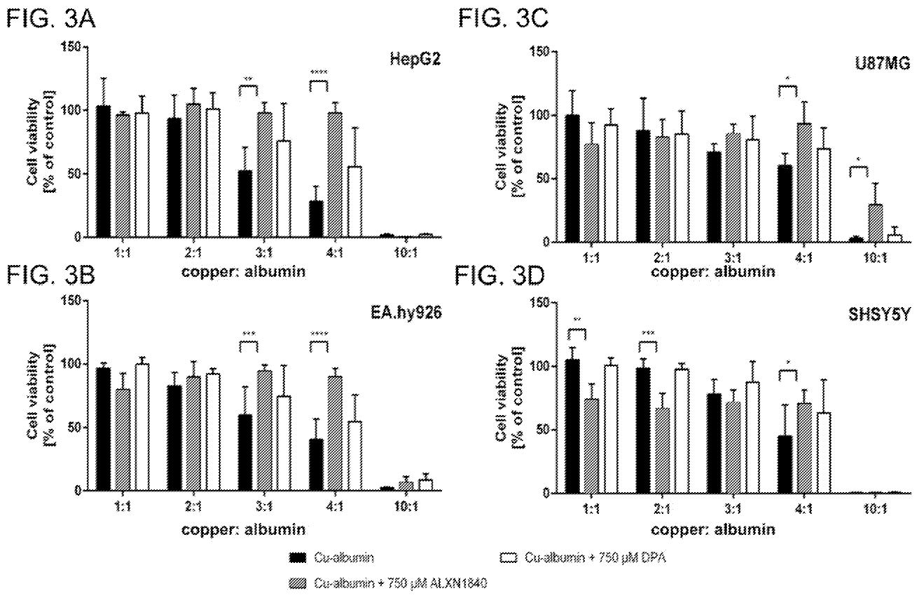

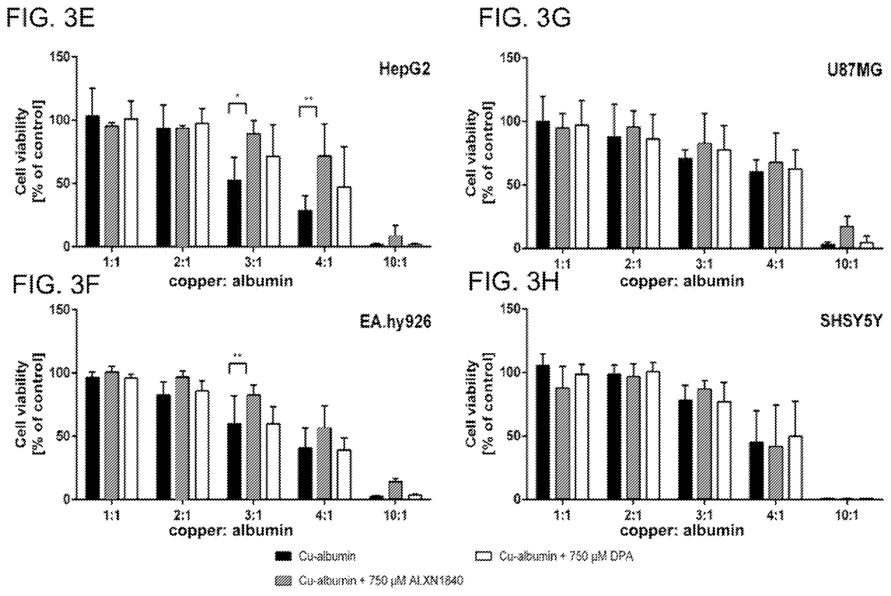

Surrogate brain cell types were tested, such as EA.hy926 (human endothelium), U87MG (human astrocytoma) and SHSY5Y (human neuroblastoma) cells, and additionally HepG2 cells (human hepatocellular carcinoma), for their vulnerability against Cu-albumin (FIG. 3). Albumin was at a concentration of 250 μM, and increasing molar ratios of Cu vs. albumin (1:1 to 10:1) were employed to mimic a progressive copper load. While at a molar ratio of 1:1, copper was rather tightly bound to albumin, at higher ratios (such as when ≥3:1) an increasing amount of loosely-bound copper was available (exemplarily shown in FIG. 2A top panel for a ratio of 3:1).

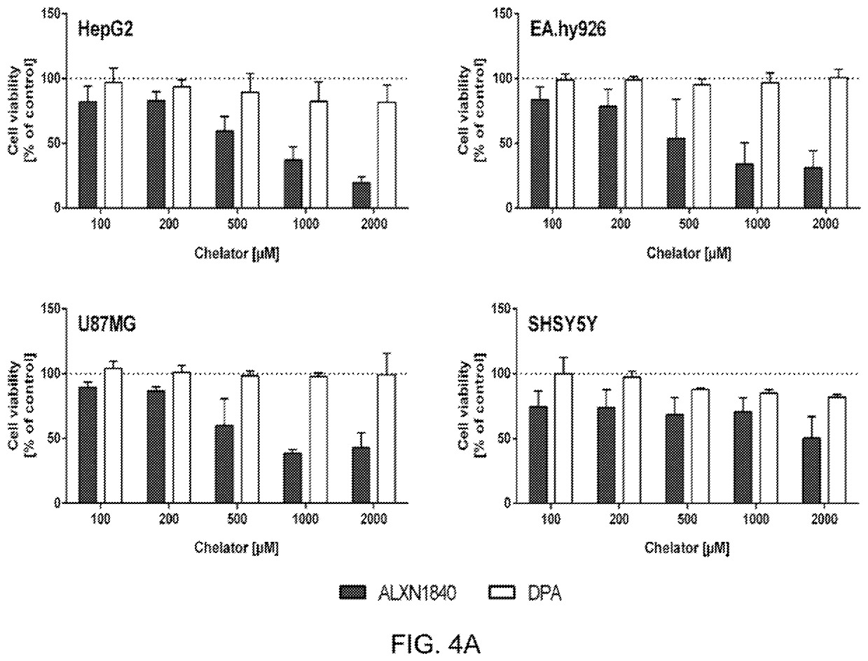

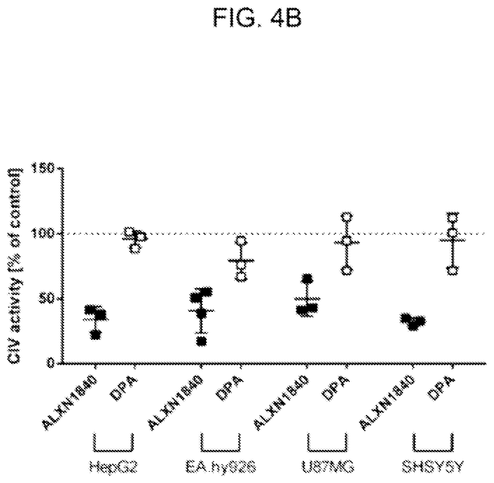

All tested cell lines demonstrated cellular toxicity against a dose-dependent increase of loosely albumin-bound Cu, as assessed by the CellTiterGlo® assay upon 24 h of incubation (FIG. 3). The high-affinity chelator ALXN1840 at a concentration of 750 μM fully avoided such toxicity up to a Cu-albumin ratio of 4:1 (FIG. 3). In contrast, DPA was much less effective. Only in HepG2 cells, and only at a high 750 μM dose, a modest rescuing effect was observed upon DPA addition (FIG. 3) that was, however, completely absent, in, e.g., endothelial EA.hy926 cells. This indicates that especially endothelial cells may be vulnerable to Cu-albumin and that DPA cannot block this Cu toxicity. Of note, the lack of DPA rescue was not due to toxicity of DPA itself, as in a copper-free setting even DPA concentrations up to 2 mM were found to be non-toxic (FIG. 4). At such settings, i.e., without external Cu-albumin addition, rather the high-affinity chelator ALXN1840 becomes toxic at elevated concentrations (FIG. 4A), possibly due to an interference with copper containing vital enzymes such as the cytochrome c oxidase (FIG. 4B).

### Example 4

### ALXN1840 Prevents Copper Toxicity by its Enormous Copper Affinity

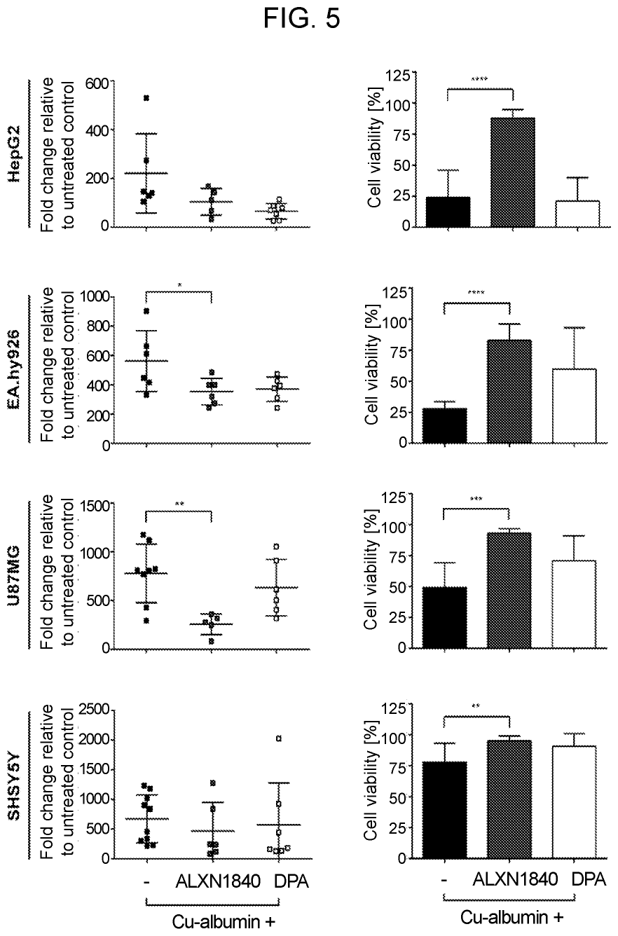

As can be seen in FIG. 5 a profound dose of Cu-albumin (here at a ratio of 3:1) increased the cellular copper content more than 100-fold in all tested cell types (FIG. 5, left panels), paralleled by massive cell death, with SHSY5Y cells being the least and EA.hy926 and HepG2 cells being the most affected (FIG. 5, right panels). This copper toxicity could not be avoided even by high doses of DPA, i.e., equimolar to copper (FIGS. 3 and 5, right panels). Moreover, in none of the cell lines a significant de-coppering was noted upon DPA co-treatment despite some tendentiously lower copper content in EA.hy926 and SHSY5Y cells (FIG. 5, left panels). In contrast, co-treatment with the high-affinity chelator ALXN1840 significantly decreased the copper content in EA.hy926 and U87MG cells, and cellular viability was significantly maintained (FIG. 5, right panels). Nevertheless, these data indicate that the cell viability protection exerted by the high-affinity chelator, however, is only in part due to its capacity to lower the cellular copper content. In HepG2 cells, for example, highly similar cellular copper contents were found in cells either co-treated by ALXN1840, DPA or treated by Cu-albumin alone (FIG. 5, upper left panel). Despite this equal copper burden, ALXN1840 rescued HepG2 cells, whereas DPA did not (FIG. 5, upper right panel). It therefore appears much more plausible that it is the enormous copper affinity of ALXN1840 (K_{d}≈10⁻²⁰) that avoided copper toxicity by its tight binding whether out—or inside cells. Indeed, even the highest known copper affinities of potential cellular binding partners are orders of magnitude lower (K_{d}≈10⁻¹⁶). In contrast, as its dissociation constant is exactly in this latter range, this may also explain why DPA was unable to ensure or only tendentiously increased cellular viability.

### Example 5

### ALXN1840 Ameliorates Cu-Albumin-Induced Mitochondrial Damage

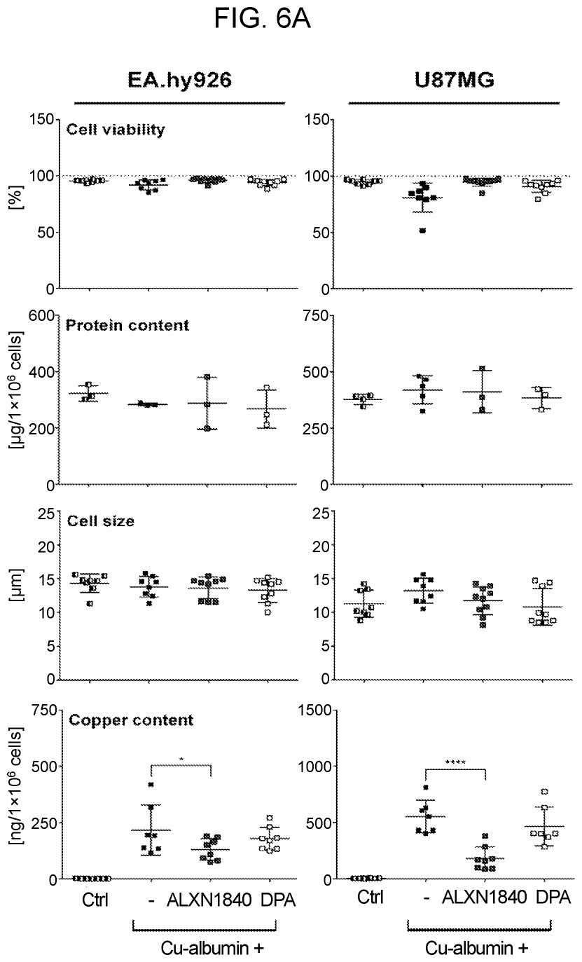

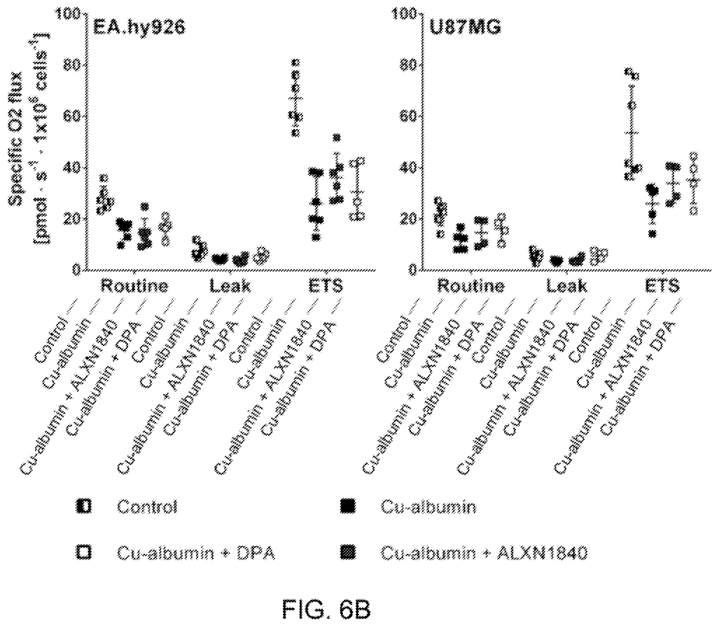

This example investigated whether Cu-albumin could impose structural and/or functional damage on mitochondria in cells that constitute the blood-brain barrier (BBB), i.e., endothelial cells and astrocytes. In order to exclude mitochondrial impairment as secondary effect of copper-induced cell demise, the Cu-albumin concentration (ratio 3:1) was adjusted such that cell viability was comparable to untreated controls (FIG. 6A). Besides, neither cellular protein content nor cell size were affected by such settings that, however, caused an enormous increase in cellular copper content with respect to untreated controls (FIG. 6A).

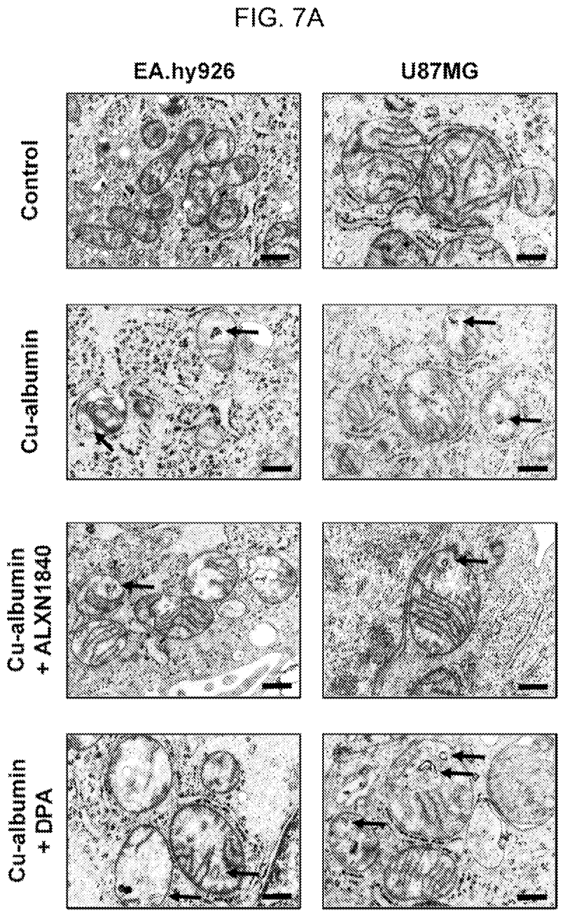

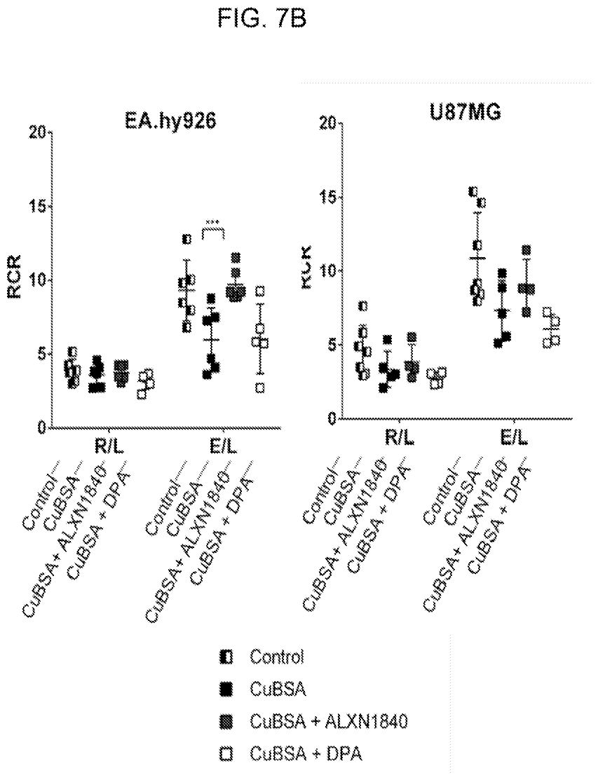

Electron micrographs of Cu-albumin vs. untreated cells demonstrated prominent mitochondrial structural alterations in EA.hy926 cells, and present, but more modest, alterations in U87MG cells (FIG. 7A). A loss or structural disorientation of the mitochondrial cristae and membranous inclusions were observed (arrows in FIG. 7A). ALXN1840 co-treatment partially avoided these structural abnormalities, demonstrating mitochondria with electron-dense matrices and structured cristae similar to untreated control cells. In contrast, DPA was of no/minor effect as mitochondria presented with short and unstructured cristae and membranous inclusions (FIG. 7A).

These structural deficits were paralleled by functional mitochondrial impairments (FIG. 7B), which were apparent in high-resolution respiratory measurements of treated cells under fully uncoupled conditions (i.e., forcing mitochondria to maximal respiration, ETS, FIG. 6B). When calculating the respiratory control ratios (the paradigm markers for mitochondrial integrity and functionality) by dividing either the “routine” (R, i.e., in presence of ADP) or the fully uncoupled state (ETS, i.e., upon FCCP titration) oxygen consumption rate by the so-called leak state (L, i.e., respiration w/o ADP), the E/L ratio demonstrated clear mitochondrial bioenergetic deficits, that again could, either significantly in the case of EA.hy926 cells or tendentiously in the case of U87MG cells, be avoided by the presence of ALXN1840, but not by DPA (FIG. 7B).

### Example 6

### Disruption of the Tight Endothelial Cell Layer of the Blood-Brain Barrier by Cu-Albumin is Prevented by ALXN1840, but not by DPA

Human EA.hy926 endothelial (and U87MG astrocytoma) cells were highly vulnerable to increasing Cu-albumin challenges (FIG. 3B) and demonstrated prominent mitochondrial structural and functional deficits (FIG. 7).

A well-characterized in vitro model of the endothelial BBB using primary porcine brain capillary endothelial cells (PBCECs) cultivated on Transwell® inserts (Muller, S. M., et al., Arch Toxicol, 2018. 92(2): p. 823-832; Hoheisel, D., et al., Biochem Biophys Res Commun, 1998. 244(1): p. 312-6) was used. As in the BBB, these primary cells form a mono-cellular tight epithelial barrier, as can be assessed by the continuous measurement of their transepithelial electrical resistance (TEER) and their monolayer capacitance as measure for cellular integrity.

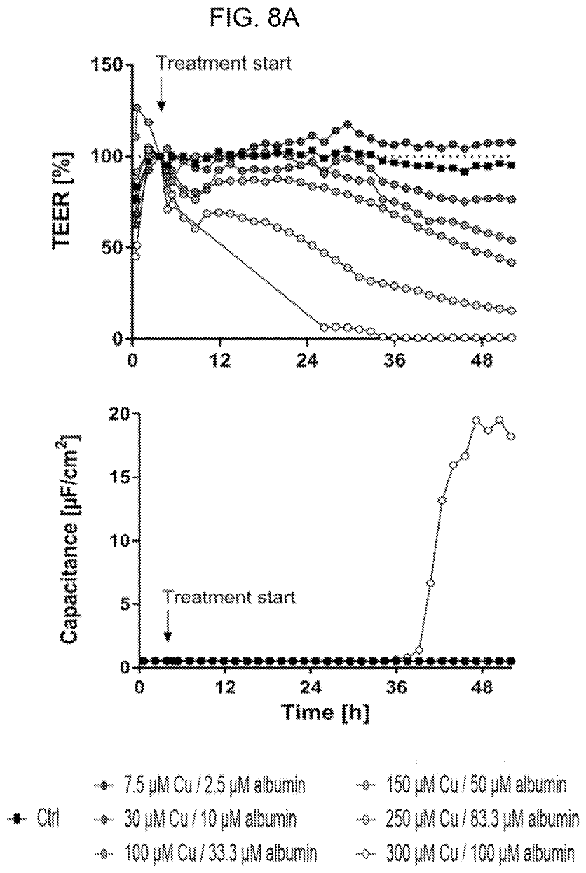

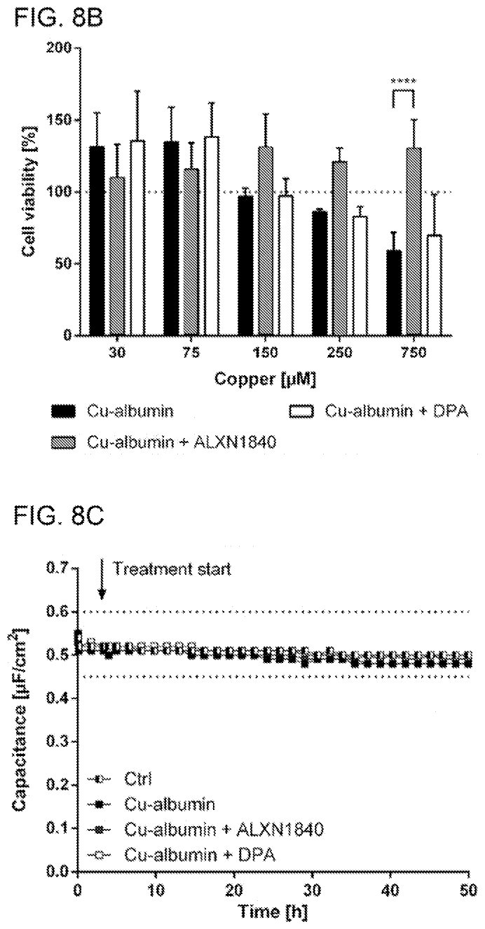

Increasing Cu-albumin concentrations (all at a molar ratio of 3:1) progressively decreased the TEER (FIG. 8A) was validated. Of note, only the highest employed Cu-albumin concentration (300 μM copper/100 μM albumin) caused a massive capacitance increase, i.e., cell death, after 36 h of incubation (FIG. 8A) that was additionally validated via the Neutral red assay (FIG. 8B). Thus, the leakiness of the endothelial cell layer of the BBB induced by lower Cu-albumin concentrations is not due to the mere induction of cell death but is a clear sign of endothelial cell stress.

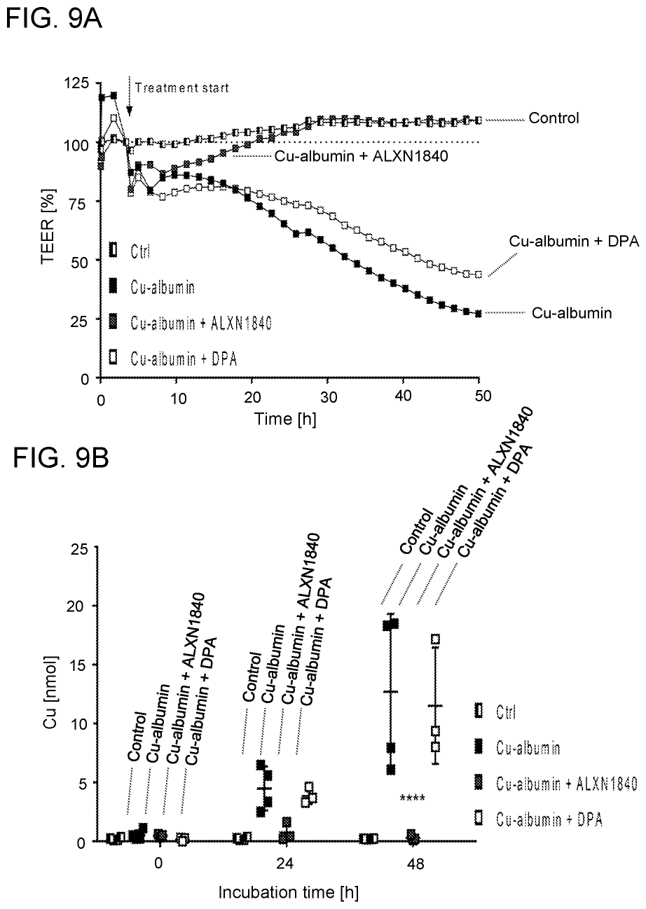

Next, the capability of the copper chelators ALXN1840 and DPA to prevent such Cu-albumin-induced endothelial BBB damage was determined (FIG. 9). A Cu-albumin concentration (250 μM Cu/83.3 μM albumin, molar ratio of 3:1) was chosen that readily caused the BBB to become leaky (i.e., decrease the TEER, FIG. 9A), but did not induce cell death within the observed time frame as evidenced by time-stable capacitance of the PBCEC monolayers (FIG. 8C). DPA co-treatment was not able to prevent the copper-induced TEER loss (FIG. 9A). In contrast, PBCEC monolayers treated with Cu-albumin in the presence of ALXN1840 demonstrated stable TEER values for 48 h, undistinguishable from untreated monolayers (FIG. 9A). This was further validated by determining the copper influx into the basolateral compartment of the Transwell® system that is not directly accessible in tight PBCEC monolayers (control in FIG. 9B). In fact, copper influx progressively occurred upon Cu-albumin treatment that could not be avoided by DPA but was fully avoided by ALXN1840 co-treatments (FIG. 9B). Thus, elevated Cu-albumin causes leakiness of the endothelial BBB layer already in the absence of endothelial cell death that is associated with copper influx into the otherwise shielded compartment, and this can be avoided by the presence of ALXN1840, but not by DPA.

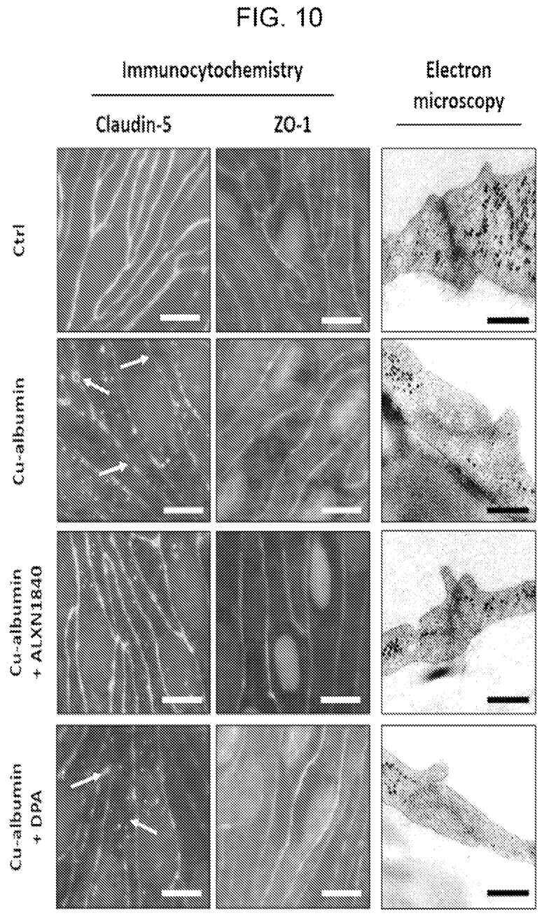

Finally, Cu-albumin-induced PBCEC monolayer damage can be visualized by either immunocytochemistry or electron microscopy (FIG. 10). First, Claudin-5, an integral membrane protein of tight junction strands, demonstrated a continuous and uninterrupted distribution at the cell margins in untreated control cells. In contrast, Cu-albumin-treated PBCECs displayed gap formations between cells as well as serrated and diffuse Claudin-5 presence. Of note, no loss/change of nuclei structure was observable validating the absence of cell death in these experiments. In the presence of ALXN1840, Claudin-5 expression was continuous and uninterrupted, whereas the presence of DPA could not prevent copper-induced gap formation (FIG. 10, left panels). Second, Zonula occludens-1 (ZO-1), an intracellular tight junction-associated protein appeared continuously present and uninterrupted at the cell borders in untreated control PBCEC monolayers (FIG. 10, middle panels). Upon Cu-albumin-treatment, a pronouncedly more diffuse staining of ZO-1 occurred that could be fully protected by ALXN1840 co-treatment, but not by DPA (FIG. 10, middle panels). Such a protein loss at the tight junctions was also apparent from, third, electron micrographs. In control PBCEC monolayers, due to deposition of the contrasting agent at protein-rich moieties, these structures appear electron-dense. Upon Cu-albumin treatment, however, these structures were much more electron permissive, not protected for by DPA, but by ALXN1840 (FIG. 10, right panels).

### Example 7

### Discussion

The present disclosure demonstrates that intravenously present copper can access the brain supporting vessels (FIG. 1) and that upon increase, copper may be progressively loosely bound to albumin (FIG. 2). Cu-albumin can be cell-toxic (FIGS. 3, 5), with endothelial cells that constitute the tight barrier to protect the brain, being especially vulnerable. But already at Cu-albumin amounts that do not exert immediate cell death, mitochondria are a vulnerable target (FIG. 7), and affected cells of the endothelial barrier demonstrate leaky tight junctions, resulting in a progressive copper cross-transition (FIGS. 9, 10). All these features were largely avoided by co-treatment with the high-affinity copper chelator ALXN1840, but not with DPA (FIGS. 2-10).

It is estimated that 18-68% of WD patients have been reported to suffer from neurological symptoms, e.g. tremor, dysarthria and dystonia. Massively elevated copper brain levels have been determined in WD patients. One possible explanation for how copper could accumulate in the brain (sometimes over decades) could be that repetitive intense pulses of copper, e.g. upon partial abrupt liver demise, transiently overwhelming the high affine plasma binding sites allowing copper uptake into brain. Indeed, in Atp7b^{-/-} rat livers, copper is not evenly distributed but rather present in “copper hotspots” with 3.5 times higher copper concentration than the surrounding liver tissue. Demise of such hotspots could possibly result in transient copper pulses. Furthermore, as ATP7B is present in the blood-facing membrane of the cerebral endothelium, mutations could lead to a reduced/blocked imminent re-transport of excessive copper into the blood, thereby causing a one-way entry of the metal into the brain parenchyma. Here only ATP7A-mediated copper transport via the CSF back into the systemic circulation would allow lowering brain copper.

The inventors aimed to mimic such copper pulses, which would result in more loosely albumin-bound copper, by testing different copper to albumin ratios. In fact, when a low ratio (1.1) was employed, no cell toxicity in any of the tested cell types was encountered. When this Cu to albumin ratio rose, however, cell viability went down, with endothelial cells being especially vulnerable. An increased mitochondrial impairment and disruption of the cellular connectivity of endothelial cell layers resembling the first barrier of the BBB, appeared as early signs of such Cu-albumin toxicity, i.e. without the initiation of cell death. Moreover, such an initial damage already allowed for progressive trans/para-epithelial copper passage. It is suggested that endothelial BBB damage upon elevated loosely protein-bound copper as one potential initial damage mechanism in neurologic WD that would subsequently allow for facilitated copper entry into brain.

Neurological worsening has been reported to frequently occur upon DPA treatment. The high-affinity copper chelator ALXN1840 caused the removal of loosely bound copper from albumin and its embedding into a deep cleft of the protein itself (termed tripartite complex TPC). Consequently, hardly any signs of Cu-albumin toxicity were encountered in the present study upon TPC formation. This may either be due to a lower cellular TPC uptake vs. loosely bound copper resulting in lower cellular copper levels, or due to a firm continuous association of copper and ALXN1840, even within cells in the studied time frames of one to two days. In contrast, DPA routed copper to the urine, demonstrating its passage into the blood and renal clearance. Such DPA-initiated copper urinal excretion was, however, quantitatively limited as only 10% of the net copper intake of Atp7b^{-/-} rats can be found in the urine upon such DPA treatment (i.e., 0.4 μmol/24 h of a total net uptake of 3.95 μmol/24 h, unpublished observation). Nevertheless, renal copper clearance by DPA occurred fast, as three days after treatment stop, elevated blood copper was not observed. As with Cu-albumin alone, hardly any beneficial effect was encountered upon DPA presence.

Neurological damage occurs frequently in WD patients. Early studies demonstrated direct copper toxicity in cat brains, but also other species. A current consensus holds that copper is the prime responsible neurotoxin in these patients. Indeed, all tested cell lines, including surrogate neuronal and astrocytic cells, are highly vulnerable to available copper. As previously reported, neurons have comparatively very low protective metallothioneins, and these cells appear relatively unprotected against copper insults. In this respect, copper-induced damage to the protective barrier cells, as demonstrated in the present study, could cause a comparatively uncontrolled copper entry into the brain. An involvement of the blood-brain barrier in four neurologic WD patients showing neurological deterioration under DPA treatment was paralleled by increased BBB damage. As rescue of the endothelial BBB by DPA co-treatment was not observed, this could have two detrimental consequences. First, neurologic WD patients with pre-damaged BBB could be highly vulnerable under DPA treatment, and second, DPA treatment could worsen pre-existing BBB damage. This suggests that neurologic patients could be tested for BBB damage (e.g., by determination of S100B levels in blood or of the albumin ratio cerebrospinal fluid/serum) before DPA treatments are initiated. It was found that such damage or leakiness occurs already at non-toxic doses and identified endothelial cell mitochondria as one vulnerable target. Mitochondria were previously described as key players in BBB permeability. In agreement with the present study, manipulation of mitochondrial respiration was paralleled with a rapid increase in BBB permeability and disruption of the tight junctions.

The blood-brain barrier is a highly sensitive structure to copper overload. Additionally, the occurrence of neurological worsening upon DPA co-treatment was linked to its inability to rescue such damage. In contrast, high-affinity chelators seem to be much more protective in this respect. Indeed, ALXN1840 was found to effectively bind loosely attached albumin copper (in this case forming the tripartite complex), and largely avoided blood-brain barrier copper toxicity.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

What is claimed is:

1. A method of reducing copper-induced neurological damage in a subject in need thereof, comprising administering to the subject a therapeutically effective amount of bis-choline tetrathiomolybdate.

2. The method of claim 1, wherein the subject has Wilson disease.

3. The method of claim 1 or claim 2, wherein reducing copper-induced neurological damage improves, reduces, or eliminates one or more neurological or psychiatric symptoms in a subject.

4. The method claim 3, wherein the one or more neurological or psychiatric symptoms is selected from: tremor, dysarthria, dystonia, gait, abnormalities, depression, social anxiety disorder, panic disorder, post-traumatic stress disorder, impairment of frontal-executive ability and/or visuospatial processing, and memory loss.

5. The method of any one of claims 1 to 4, wherein reducing copper-induced neurological damage is by formation of stable Cu-tetrathiomolybdate albumin tripartite complexes.

6. The method of claim 6, wherein the Cu-tetrathiomolybdate albumin tripartite complexes are available in the systemic circulation in the subject for transportation and/or elimination.

7. The method of any one of claims 1 to 6, wherein the copper-induced neurological damage comprises one or more: copper-induced cell toxicity, copper-induced blood-brain barrier damage, and copper-induced mitochondrial damage.

8. The method of any one of claims 1 to 6, wherein reducing copper-induced neurological damage is by reducing copper-induced cell toxicity.

9. The method of claim 8, wherein the copper-induced cell toxicity is reduced in at least one of liver cells, endothelial cells, neurons, and astrocytes.

10. The method of claim 8 or claim 9, wherein reducing copper-induced cell toxicity improves cell viability (as compared to the cell viability without bis-choline tetrathiomolybdate administration).

11. The method of any one of claims 1 to 6, wherein reducing copper-induced neurological damage is by reducing copper-induced blood-brain barrier damage.

12. The method of claim 11, wherein reducing of the copper-induced blood-brain barrier damage comprises one or more of: (a) maintaining transepithelial electrical resistance (TEER), (b) maintaining capacitance, and (c) reducing copper-induced morphological alterations.

13. The method of claim 12, wherein TEER is maintained at the same level as in a healthy subject.

14. The method of claim 12 or claim 13, wherein capacitance is maintained at the same level as in a healthy subject.

15. The method of any one of claims 12 to 14, wherein the copper-induced morphological alteration is a loss or structural disorientation of mitochondrial cristae and/or membranous inclusions.

16. The method of any one of claims 12 to 15, wherein reducing copper-induced morphological alteration results in continuous and uninterrupted presence of Claudin-5 and/or Zonula Occludens-1 presence at the cell borders of brain capillary endothelial cells.

17. The method of any one of claims 1 to 6, wherein reducing copper-induced neurological damage is by reducing copper-induced mitochondrial damage.

18. The method of claim 17, wherein reducing of the copper-induced mitochondrial damage comprises one or more of: (a) reducing the presence of copper-induced membrane inclusions, (b) increasing or maintaining cristae organization, (c) increasing or maintaining electron-dense matrices, and (d) increasing or maintaining mitochondrial respiration, all as compared to untreated subject.

19. The method of any one of claims 1 to 17, wherein reducing copper-induced neurological damage occurs within a time interval ranging from day 0 to week 24 post administration.

20. The method of claim 19, wherein the highest level of reduction is within a interval ranging from day 0 to week 7 post administration.

21. The method of any one of claims 1 to 20, wherein the subject previously received no treatment for the copper metabolism-associated disease or disorder, such as for Wilson disease (i.e., a treatment-naïve subject).

22. The method of any one of claims 1 to 20, wherein the subject previously received a standard of care treatment for the copper metabolism-associated disease or disorder, such as for Wilson disease.

23. The method of claim 22, wherein the standard of care treatment comprises trientine, D-penicillamine, and/or zinc.

24. The method of claim 22, wherein the standard of care treatment comprises D-penicillamine.

25. The method of any one of claims 1 to 24, wherein the therapeutically effective amount of bis-choline tetrathiomolybdate is in the range of about 15 mg to about 60 mg per day.

26. The method of any one of claims 1 to 24, wherein the therapeutically effective amount of bis-choline tetrathiomolybdate is about 15 mg daily.

27. The method of any one of claims 1 to 24, wherein the therapeutically effective amount of bis-choline tetrathiomolybdate is about 30 mg daily.

28. The method of any one of claims 1 to 24, wherein the therapeutically effective amount of bis-choline tetrathiomolybdate is about 15 mg every other day.

29. The method of any one of claims 1 to 24, wherein the therapeutically effective amount of bis-choline tetrathiomolybdate is about 30 mg every other day.

30. The method of any one of claims 1 to 29, further comprising adjusting the therapeutically effective amount of bis-choline tetrathiomolybdate to a second therapeutically effective amount of bis-choline tetrathiomolybdate based on the change in one or more neurological or psychiatric symptoms.

31. The method of claim 30, wherein the second therapeutically effective amount of bis-choline tetrathiomolybdate is lower than the first therapeutically effective amount of bis-choline tetrathiomolybdate.

32. The method of claim 30, wherein the second therapeutically effective amount of bis-choline tetrathiomolybdate is higher than the first therapeutically effective amount of bis-choline tetrathiomolybdate.

33. A composition comprising a therapeutically effective amount of bis-choline tetrathiomolybdate for use in the methods any one of claims 1 to 24 and 30 to 32.

34. The composition of claim 33, wherein the therapeutically effective amount of bis-choline tetrathiomolybdate is in the range of about 15 mg to about 60 mg per day.

35. The composition of claim 33, wherein the therapeutically effective amount of bis-choline tetrathiomolybdate is about 15 mg daily.

36. The composition of claim 33, wherein the therapeutically effective amount of bis-choline tetrathiomolybdate is about 30 mg daily.

37. The composition of claim 33, wherein the therapeutically effective amount of bis-choline tetrathiomolybdate is about 15 mg every other day.

38. The composition of claim 33, wherein the therapeutically effective amount of bis-choline tetrathiomolybdate is about 30 mg every other day.
